SEQUENCE LISTING (15 sequences)

SEQ ID NO: 1 - DNA, 669 bp, Severe acute respiratory syndrome coronavirus 2
  source 1..669: mol_type = genomic DNA
atggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaa

SEQ ID NO: 2 - DNA, 40 bp, synthetic construct
  source 1..40: mol_type = other DNA
taatacgactcactatagtaatcagacaaggaactgatta

SEQ ID NO: 3 - DNA, 19 bp, synthetic construct
  source 1..19: mol_type = other DNA
cgaaggtgtgacttccatg

SEQ ID NO: 4 - DNA, 18 bp, synthetic construct
  source 1..18: mol_type = other DNA
ttgctatcgcaatggctt

SEQ ID NO: 5 - DNA, 18 bp, synthetic construct
  source 1..18: mol_type = other DNA
tgatgtcacagcgtccta

SEQ ID NO: 6 - DNA, 42 bp, synthetic construct
  source 1..42: mol_type = other DNA
ggagtggcacgttgagaagaatgcttctttcagactgtttgc

SEQ ID NO: 7 - DNA, 40 bp, synthetic construct
  source 1..40: mol_type = other DNA
ggcactattctgaccagaccggatgtccacgaaggatcac

SEQ ID NO: 8 - DNA, 22 bp, synthetic construct
  source 1..22: mol_type = other DNA
ggattgaatgaccacatggaac

SEQ ID NO: 9 - DNA, 20 bp, synthetic construct
  source 1..20: mol_type = other DNA
agtgaactcgtaatcggagc

SEQ ID NO: 10 - DNA, 20 bp, synthetic construct
  source 1..20: mol_type = other DNA
gcttctttcagactgtttgc

SEQ ID NO: 11 - DNA, 22 bp, synthetic construct
  source 1..22: mol_type = other DNA
ggagtggcacgttgagaagaat

SEQ ID NO: 12 - DNA, 19 bp, synthetic construct
  source 1..19: mol_type = other DNA
gatgtccacgaaggatcac

SEQ ID NO: 13 - DNA, 21 bp, synthetic construct
  source 1..21: mol_type = other DNA
ggcactattctgaccagaccg

SEQ ID NO: 14 - DNA, 20 bp, synthetic construct
  source 1..20: mol_type = other DNA
tcttctcaacgtgccactcc

SEQ ID NO: 15 - DNA, 20 bp, synthetic construct
  source 1..20: mol_type = other DNA
ccttgatgtcacagcgtcct

(19) United States
(12) Patent Application Publication
(10) Pub. No.: US 2026/0209834 A1
(43) Pub. Date: Jul. 23, 2026

(54) NUCLEIC ACID DETECTION

(71) Applicant: UNIVERSITY OF SURREY, Guildford, Surrey (GB)

(72) Inventors: Khushboo BORAH SLATER, Guildford, Surrey (GB); Johnjoe MCFADDEN, Guildford, Surrey (GB); Roberto LA RAGIONE, Guildford, Surrey (GB); Ravi SILVA, Guildford, Surrey (GB); Muhammad AHMAD, Guildford, Surrey (GB); Aurore POIRIER, Guildford, Surrey (GB)

(21) Appl. No.: 19/129,448

(22) PCT Filed: Nov. 14, 2023
(86) PCT No.: PCT/GB2023/052976

§ 371 Date: May 13, 2025

(30) Foreign Application Priority Data
Nov. 15, 2022 (GB) 2217029.4

(51) Int. Cl. C12Q 1/6848; C12Q 1/689; C12Q 1/70; G01N 27/30; G01N 27/327; G01N 27/48

(52) U.S. Cl. CPC C12Q 1/6848; C12Q 1/689; C12Q 1/701; G01N 27/308; G01N 27/3278; G01N 27/48; C12Q 2600/156

(57) ABSTRACT

The invention relates to nucleic acid detection, and particularly, although not exclusively, to electrochemical methods for detecting target nucleic acid sequences in a sample obtained from a subject. The nucleic acid detection methods of the invention can also be used to diagnose or prognose both infectious and non-infectious diseases in a subject, or to determine whether a pathogen is drug resistant. The invention extends to apparatus for performing improved nucleic acid detection using electrochemical methods.

The present invention relates to nucleic acid detection, and particularly, although not exclusively, to electrochemical methods for detecting target nucleic acid sequences in a sample obtained from a subject. The nucleic acid detection methods of the invention can also be used to diagnose or prognose both infectious and non-infectious diseases in a subject, or to determine whether a pathogen is drug resistant. The invention extends to apparatus for performing improved nucleic acid detection using electrochemical methods.

During the COVID-19 pandemic, it has become clear that detecting and tracing infection cases is critical for controlling the spread of viral transmission. Additionally, with the increase in antibiotic resistance, it is increasingly important to determine whether an individual is infected with an antibiotic resistant bacteria, in order to provide an effective treatment. For this reason, there is a significant need for an accurate, rapid and sensitive method for detecting target nucleic acid sequences in clinical samples.

Both polymerase chain reaction (PCR) and loop mediated isothermal amplification (LAMP) are relatively rapid, accurate, and cost-effective nucleic acid amplification techniques with high specificity and sensitivity. LAMP is an isothermal reaction method, and the target amplification is performed at DNA melting temperature (60-65° C.) with a specialised DNA polymerase (Bst DNA polymerase) and several sets of primers. Since the reaction produces pyrophosphates, the pH change can be used as a readout. Alternatively, fluorescent staining of dsDNA or electrophoresis of amplicons can be used to identify LAMP products. PCR is a similar technique but requires the sample to be cycled through several temperatures.

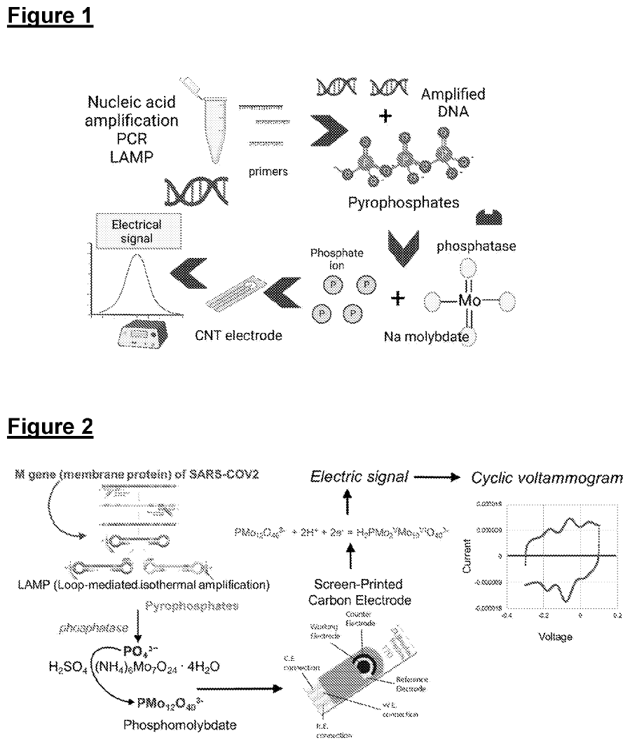

It has also been shown that electrochemical quantification of genomic DNA from Nosema bombycis, an intracellular parasite, can be achieved by tracing phosphate ions (Pi) generated during the LAMP process. During the LAMP reaction, one mole of deoxyribonucleotide monophosphate incorporated into dsDNA generates one mole of pyrophosphate (PPi). In the presence of thermostable inorganic pyrophosphatase (PPase), the produced PPi is then hydrolysed into phosphate ions (Pi), which can further react with acidic molybdate to form molybdophosphate on the electrode surface afterwards. The electrochemical response of the generated molybdophosphate can then be detected for target quantitative analysis with high sensitivity. This electrochemical detection principle of tracing Pi ions generated during LAMP to form molybdophosphate for signal output is illustrated in FIG. 1.

However, despite the finding that electrochemical quantification of N. bombycis genomic DNA can be achieved by tracing phosphate ions (Pi) generated during the LAMP process, it has not been possible to apply this method to clinical samples in order to diagnose diseases in humans.

There is, therefore, a need to provide an improved assay for quantitatively detecting target nucleic acids found in clinical samples, with high sensitivity and accuracy.

When detecting target nucleic acid sequences in clinical samples using the electrochemical method, the inventors found that false positive results were produced. However, the cause of such false positives was initially unknown. The inventors then surprisingly discovered that these false positives were caused by the presence of free phosphate ions within the clinical samples. Accordingly, in view of this surprising discovery, the inventors decided to modify the assay in order to reliably and accurately detect target nucleic acids in clinical samples obtained from patients using an electrochemical signal.

Therefore, according to a first aspect of the invention, there is provided a method of detecting a target nucleic acid sequence in a sample obtained from a subject, the method comprising:
removing substantially all free phosphate ions from a sample comprising a target nucleic acid sequence;
amplifying the target nucleic acid sequence in the sample to generate amplified products;
using the amplified products to create an electroactive compound; and
detecting an electrochemical signal of the electroactive compound, wherein an electrochemical signal indicates the presence of the target nucleic acid sequence in the sample.

According to a second aspect of the invention, there is provided a method of diagnosing or prognosing a disease in a subject, the method comprising detecting a target nucleic acid sequence in a sample obtained from a subject, by:
removing substantially all free phosphate ions from a sample comprising a target nucleic acid sequence;
amplifying the target nucleic acid sequence in the sample to generate amplified products;
using the amplified products to create an electroactive compound; and
detecting an electrochemical signal of the electroactive compound, wherein an electrochemical signal indicates that the subject suffers from a disease, is infected by a targeted pathogen, or has a negative prognosis.

According to a third aspect of the invention, there is provided an apparatus for detecting a target nucleic acid sequence in a sample obtained from a subject, the apparatus comprising:
means for removing substantially all free phosphate ions from a sample comprising a target nucleic acid sequence;
means for amplifying the target nucleic acid sequence in the sample to generate amplified products;
means for creating an electroactive compound using the amplified products; and
means for detecting an electrochemical signal of the electroactive compound.

As discussed above, the inventors were surprised to observe that free phosphate ions in clinical samples were interfering with electrochemical detection, leading to false positive results. By removing the free phosphate ions from biological fluids or clinical samples before carrying out the assay, the inventors have been the first to demonstrate that an electrochemical assay can be used to accurately detect target nucleic acid sequences in clinical samples, in particular for the diagnosis of infectious diseases in a subject.

The inventors were surprised to discover that when performing the electrochemical assay on clinical samples, the presence of free phosphate ions was leading to false positive results. Accordingly, to avoid phosphate interference and reduce background noise, the inventors performed a sample clean-up step to remove the free phosphate ions from the biological fluid or clinical sample.

It is well-known to the skilled person that “free phosphate ions” are those which are not bound by another molecule and are free in the sample or solution. Free phosphate ions may also be referred to as inorganic phosphate, PO₄³⁻ (Pi), and may also include pyrophosphate.

In one embodiment, removing substantially all free phosphate ions from the sample preferably comprises removing at least 50%, at least 60%, or at least 70% of the free phosphate ions from the sample. Preferably, at least 80%, at least 90%, or at least 95% of the free phosphate ions are removed from the sample. More preferably, at least 96%, at least 97%, at least 98%, or at least 99% of the free phosphate ions are removed from the sample. Alternatively, in another embodiment, the method comprises removing 100% of the free phosphate ions from the sample. Preferably, the free phosphate ions are removed from the sample prior to the amplification step.

There are many methods that can be employed to remove free phosphate ions from biological or clinical samples, and these will be well-known to the skilled person.

In one embodiment, the free phosphate ions maybe removed from the sample using phosphate-removing bacteria. In one embodiment, the phosphate-removing bacteria are selected from Acinetobacter johnsonii 210A, Acinetobacter lwoffii, Pseudomonas aeruginosa AS1, Moraxella lacunata AS2, and Alcaligenes denitrificans AS4.

Alternatively, in another embodiment, the free phosphate ions may be removed from the sample by selective absorption. For example, the free phosphate ions may be removed by passing the samples over red mud (a waste residue of alumina refinery), aluminium hydroxide, or zirconium or lanthanum-based materials.

Alternatively, in another embodiment, the free phosphate ions are removed from the sample by ferric trichloride hexahydrate (FeCl₃) precipitation. Preferably, the FeCl₃ precipitation method comprises adding potassium phosphate (KH₂PO₄) to water (preferably, type 1 water). Preferably, the method comprises adding FeCl₃ to the KH₂PO₄ solution. Preferably, the method comprises stirring the solution and/or adjusting the pH to 7.0. Preferably, the solution is withdrawn after a period of time from the start of the assay, for example, 10 minutes, 20 minutes, 24 h, 48 h, 72 h or 144 h. The withdrawn samples are preferably centrifuged (e.g. at 13000 rpm for 5 minutes) and the supernatant is filtered. Preferably, the amount of phosphate remaining in the filtered supernatant is measured using colorimetric molybdenum blue assay.

Most preferably, however, the free phosphate ions are removed from the sample using a phosphate removal column. Preferably, the phosphate removal column comprises a resin that binds phosphates in buffers and samples. Preferably, the phosphate binding capacity of the resin is about 0.20 mmole per milliliter of the bed volume. Preferably, the phosphate removal column is spun using a microcentrifuge to set down the resin. Preferably, the phosphate removal column is spun at about 13,000 rpm for at least 1 minute and the solution is discarded. Preferably, the phosphate removal column is spun at about 13,000 rpm for at least 1 minute to make sure the resin is almost dry. Preferably, the phosphate removal column is transferred into an eppendorf tube. Preferably, the sample is loaded onto the column and spun at about 1000 rpm for at least 1 minute. Preferably, the column is spun again at about 13,000 rpm for at least 1 minute and the elute is saved. Preferably, the elute is spun at about 13,000 rpm for at least 1 minute to remove any insoluble material.

It will be appreciated that the apparatus according to the third aspect may comprise any one of the above-specified means for removing free phosphate ions from the sample.

In one embodiment, the method may further comprise measuring the concentration of free phosphate ions in the sample after substantially all the free phosphate ions have been removed. For example, the method may comprise detecting free phosphate ions using the molybdenum blue phosphate reaction.

It will be well understood by the skilled person that the molybdenum blue reaction involves the formation of a polyoxometallate species, a heteropoly acid, from orthophosphate and molybdate under acidic conditions, which when reduced forms an intensely coloured phosphomolybdenum blue species. Preferably, the molybdenum blue reaction comprises preparing solutions of sulphuric acid (11N/5.4M), ascorbic acid (6% w/v), ammonium molybdate tetrahydrate (1.26% w/v)-potassium antimony tartrate trihydrate (0.21% w/v) and potassium phosphate monobasic (0.1M) in distilled water from concentrated stock solutions. Preferably, potassium dihydrogen phosphate (KH₂PO₄) standards are prepared in a range of dilutions in distilled water. Preferably, working solution of sulphuric acid, ammonium molybdate-potassium tartrate and ascorbic acid solution working solution are added to the samples. Preferably, the reaction is incubated at room temperature for 5 minutes. Preferably, the reaction is transferred to a cuvette and absorbance is measured (at 650 nm) using a spectrophotometer.

Amplification techniques for amplifying the target nucleic acid sequence are well known to the skilled person and include, but are not limited to, polymerase chain reaction (PCR), loop-mediated isothermal amplification (LAMP), recombinase polymerase amplification (RPA), nucleic acid sequence-based amplification (NASBA), and the like.

Thus, in one embodiment, the amplification step may comprise amplifying the target nucleic acid sequence in the sample by PCR to generate PCR products.

PCR is a standard, well-known technique for nucleic acid amplification. The PCR may be Quantitative (Q)-PCR, droplet-digital PCR and Crystal™ Digital™ PCR. All of these techniques are routine in molecular biology and known to those skilled in the art.

Alternatively, in another embodiment, the amplification step may comprise amplifying the target nucleic acid sequence by LAMP to generate LAMP products.

Loop-mediated isothermal amplification (LAMP) is a single-step, isothermal amplification reaction utilising a DNA polymerase with strand displacement activity. The amplification reaction produces a stem-loop DNA with multiple inverted repeats of the target nucleic acid sequence.

It is well-known to the skilled person that nucleic acid extraction may take place prior to amplification (e.g. by LAMP or PCR). Accordingly, in a preferred embodiment, the method further comprises extracting and/or purifying the nucleic acid from the sample before the amplification step. Preferably, the apparatus comprises means for extracting and/or purifying the nucleic acid from the sample.

In a preferred embodiment, amplifying the target nucleic acid sequence in the sample (e.g. by LAMP or PCR) comprises contacting the sample with one or more primers.

The term “primer” designates, within the context of the present invention, a nucleotide sequence that can hybridize specifically to the target nucleic acid sequence and serve to initiate amplification.

Preferably, when the amplification step comprises amplification by PCR, the one or more primers comprises a forward primer and a reverse primer. The forward primer is designed so that it is complementary to a sequence of nucleotides upstream of the sequence of interest, whilst the reverse primer is designed so that it is complementary to a sequence of nucleotides downstream of the sequence of interest.

Preferably, when the amplification step comprises amplification by LAMP, the one or more primers comprises primers selected from a group consisting of: a forward loop primer (LF); a backward loop primer (LB); a forward outer primer (F3); a backward outer primer (B3); a forward inner primer (FIP); and a backward inner primer (BIP).

In some embodiments, the amplification may be multiplexed. Advantageously, this allows the simultaneous detection of multiple target nucleic acids within the same reaction.

Accordingly, in one embodiment, the amplification step comprises contacting the sample with two or more sets of primers, wherein each set of primers are distinct for each target nucleic acid to be amplified. Preferably, when the multiplexed amplification is amplification by PCR, each set of primers comprises a forward primer and a reverse primer. Preferably, when the multiplexed amplification is amplification by LAMP, each set of primers comprises one or more primers selected from a group consisting of: a forward loop primer (LF); a backward loop primer (LB); a forward outer primer (F3); a backward outer primer (B3); a forward inner primer (FIP); and a backward inner primer (BIP). Preferably, for multiplexed LAMP amplification, the amplification step comprises the use of at least a pair of primers selected from: an LF and LB pair; an F3 and B3 pair; and an FIP and BIP pair.

In a preferred embodiment, amplifying the target nucleic acid sequence in the sample (e.g. by LAMP or PCR) comprises contacting the sample with a polymerase. In one embodiment, the polymerase is a DNA polymerase. The DNA polymerase may be selected from a group consisting of: Bacillus stearothermophilus (Bst) polymerase; Bacillus smithii (Bsm) polymerase; and Taq polymerase. Preferably, when the amplification step is amplification by LAMP, the DNA polymerase is Bacillus stearothermophilus (Bst) polymerase. Preferably, when the amplification step is amplification by PCR, the DNA polymerase is Taq polymerase.

In a preferred embodiment, amplifying the target nucleic acid sequence in the sample (e.g. by LAMP or PCR) comprises contacting the sample with a plurality of deoxynucleotide triphosphates (dNTPs). Preferably, the plurality of deoxynucleotide triphosphates (dNTPs) are selected from the group consisting of dATP, dGTP, dCTP and/or dTTP. dNTPs are the building blocks of DNA.

It is well-known to the skilled person that a typical amplification reaction (e.g. by LAMP or PCR) generates pyrophosphates (PPi) which are proportional to the amount of amplified target nucleic acid. Accordingly, as used herein, the terms “amplified products”, “PCR products” and/or “LAMP products” refer to the amplified target nucleic acid and pyrophosphates (PPi).

Thermostable pyrophosphatase (PPase) can be used in the reaction buffer to cleave the pyrophosphates (PPi) into phosphates (Pi) during the amplification reaction. Accordingly, in a preferred embodiment, the amplification step further comprises contacting the amplified products with pyrophosphatase (PPase). The pyrophosphatase converts pyrophosphates (PPi) generated by the amplification reaction into phosphate ions (Pi).

Reverse transcriptase may be added to the reaction for amplification of RNA target sequences. For example, reverse transcriptase-LAMP (RT-LAMP) or reverse transcription PCR (RT-PCR) may be used to amplify RNA target sequences. Accordingly, in one embodiment, the amplification step comprises contacting the sample with reverse transcriptase.

In one embodiment, when the amplification is by PCR, the amplification step further comprises contacting the sample with buffer and/or water. Preferably, the buffer comprises Tris HCl, KCl, and/or MgCl₂.

Preferably, the one or more primers, the plurality of dNTPs, the polymerase, the pyrophosphatase, the reverse transcriptase, the buffer, and/or water are pre-mixed to form the “PCR reaction buffer”.

In one embodiment, when the amplification is by LAMP, the amplification step further comprises contacting the sample with isothermal amplification buffer, MgSO₄, DMSO, phosphatase, water and/or template sRNA. Preferably, the isothermal amplification buffer comprises Tris-HCl, ammonium sulphate (NH₄)₂SO₄, potassium chloride KCl, magnesium sulphate MgSO₄ and/or Tween® 20.

Preferably, the one or more primers, the plurality of dNTPs, the polymerase, the pyrophosphatase, the reverse transcriptase, the isothermal amplification buffer, MgSO₄, DMSO, phosphatase, water and/or template sRNA are pre-mixed to form the “LAMP reaction buffer”.

It will be appreciated that the apparatus according to the third aspect, may comprise any of the above specified reagents as means for amplifying the target nucleic acid sequence in the sample (e.g. by LAMP or PCR).

Preferably, when the amplification is by PCR, the amplification step comprises the activation of DNA polymerase and initial denaturation. Preferably, the initial denaturation is carried out at a temperature of between 92 and 98° C., between 93 and 97° C., or between 94 and 96° C. Preferably, the activation of DNA polymerase and initial denaturation is carried out for a period of between 30 seconds and 5 minutes, or between 1 and 4 minutes, or between 2 and 3 minutes.

Preferably, PCR is carried out for between 20 and 40 cycles, or between 25 and 35 cycles. Preferably, the cycle includes the following steps:
denaturation;
annealing; and
extension.

Preferably, the denaturation step of PCR is carried out at a temperature of between 92 and 98° C., between 93 and 97° C., or between 94 and 96° C. Preferably, the denaturation step is carried out for a period of between 10 seconds and 2 minutes, between 20 seconds and 1 minute, or between 30 seconds and 1 minute.

Preferably, the annealing step of PCR is carried out at a temperature of between 40 and 6W° C., or between 45 and 55° C. Preferably, the annealing step is carried out for a period of between 20 seconds and 2 minutes, between 20 seconds and 1 minute, or between 30 seconds and 1 minute.

Preferably, the extension step of PCR is carried out at a temperature of between 66 and 78° C., between 68 and 76° C., or between 7° and 74° C. Preferably, the extension step is carried out for a period of time between 30 seconds and 3 minutes, between 30 seconds and 2 minutes, or between 30 seconds and 1 minute.

Preferably, PCR includes a final extension step. Preferably, the final extension step is carried out at a temperature of between 62 and 74° C., between 64 and 72° C., or between 66 and 70° C. Preferably, the final extension is carried out for a period of time between 5 and 15 minutes, between 6 and 14 minutes, between 7 and 13 minutes, between 8 and 12 minutes, or between 9 and 11 minutes.

In a preferred embodiment, when the amplification is by LAMP, the amplification step is conducted at a temperature of between 5° and 75° C., between 52 and 73° C., between 54 and 71° C., between 56 and 69° C., between 58 and 67° C., or between 6° and 65° C.

In one embodiment, when the amplification is by LAMP, the amplification step is conducted at a temperature of between 5° and 73° C., between 5° and 71° C., between 5° and 69° C., between 5° and 67° C., or between 5° and 65° C. In another embodiment, when the amplification is by LAMP, the amplification step is conducted at a fixed temperature of between 52 and 75° C., between 54 and 75° C., between 56 and 75° C., between 58 and 75° C., or between 6° and 75° C. Most preferably, when the amplification is by LAMP, the amplification step is conducted at a temperature of about 65° C.

In one embodiment, when the amplification is by LAMP, the amplification step is conducted for between 10 and 60 minutes, between 15 and 50 minutes, between 20 and 40 minutes, or between 25 and 35 minutes. Preferably, when the amplification is by LAMP, the amplification step is conducted for about 30 minutes.

Phosphate ions, when added to a phosphate-precipitating salt such as sodium molybdate, produce phosphomolybdate, which in turn undergoes oxidation and reduction reactions and generates an electric current under acidic conditions.

Accordingly, using the amplified products to create an electroactive compound (comprising an electrochemical current, voltage or signal) preferably comprises 35 contacting the amplified products (the phosphate ions) with a phosphate-precipitating salt. Preferably, the phosphate-precipitating salt comprises or is a molybdate, more preferably a Group I metal molybdate. Preferably, the phosphate-precipitating salt is sodium molybdate. The electrochemical current can be conducted along an electrode, and then subsequently detected. Thus, preferably the step comprises conducting the electrochemical signal along an electrode.

It will be appreciated that the phosphate ions react with the phosphate-precipitating salt (preferably sodium molybdate) to produce phosphomolybdate.

In one embodiment, the amplified products and phosphate-precipitating salt (preferably sodium molybdate) are mixed prior to contacting with the electrode. Preferably, the amplified products and phosphate-precipitating salt (preferably sodium molybdate) are mixed by pipetting. In a preferred embodiment, the amplified products and phosphate-precipitating salt (preferably sodium molybdate) are mixed in a ratio from about 1:10 to about 10:1. Most preferably, the amplified products and phosphate-precipitating salt (preferably sodium molybdate) are mixed in a ratio of 1:1. Preferably, the mixture is incubated at room temperature. Preferably, the mixture is incubated for between 5 and 40 minutes, or between 10 and 30 minutes, or more preferably between 15 and 20 minutes.

In one embodiment, the electrochemical signal is detected using a two electrode system comprising a working electrode (WE) and a counter electrode (CE), or more preferably using a three electrode system comprising a WE, a CE and a reference electrode (RE). In one embodiment, the electrode is in the form of a conventional electrochemical cell. Alternatively, in another embodiment, the electrode is in the form of a micro-electrode array fabricated on an insulating rigid/flexible substrate. More preferably, the electrode is in the form of screen-printed electrodes on an insulating rigid/flexible substrate. Preferably, the electrode is made of an electrically conducting material. For example, the material of the electrode may be selected from the group consisting of: C (carbon), Au, Ag, Pt, Pd, Cr, Ti, Ni, W, Ta, Co, Ru, graphene, graphene oxide (GO), reduced graphene oxide (rGO), Carbon nanotubes (CNTs), carbon fibres, fullerene, ITO, and PEDOT, or a combination of two or more such materials. Preferably, the electrode is a carbon based screen-printed electrode. More preferably, the electrode is a carbon nanotube (CNT) modified carbon based screen-printed electrode. Most preferably, the electrode is an acid or O₂ plasma functionalised CNT modified carbon-based screen printed electrode. Preferably, the electrode is connected to a potentiostat for measuring the electrochemical signal.

In one embodiment, the electrode may be rinsed with ultra-pure water prior to being contacted with the amplified products and phosphate-precipitating salt (preferably sodium molybdate).

In another embodiment, the electrode is primed with sodium nitrate prior to being contacted with the amplified products and phosphate-precipitating salt (preferably sodium molybdate). Preferably, the electrode is primed with sodium nitrate for between 3 and 10 cycles.

In one embodiment, the method further comprises drying the electrode after it has been contacted with the amplified products and phosphate-precipitating salt (preferably sodium molybdate). Preferably, drying the electrode comprises drying at room temperature, or drying in an oven at a temperature of between 25 and 100° C., between 30 and 90° C., between 35 and 80° C., between 40 and 70° C. and between 46 and 60° C. Most preferably, drying the electrode comprises drying in an oven at a temperature of about 50° C.

Preferably, drying the electrode comprises drying in an oven for between three and five minutes. Most preferably, drying the electrode comprises drying in an oven at a temperature of about 50° C. for about five minutes.

In another embodiment, the method further comprises rinsing the electrode with ultra-pure water after it has been dried.

In one embodiment, the method further comprises contacting the electrode with an electrolyte. Preferably, this step take place after the electrode has been dried and rinsed with ultra-pure water. The electrolyte may be selected from solutions of salts, acids or bases. Examples of electrolytes include alkali metal salts of nitrate, borate, halides, phosphate, phosphonates, carbonate, sulphate; acids such as sulphuric acid (H₂SO₄), phosphoric acid (H₃PO₄), perchloric acid (HClO₄), or bases such as potassium hydroxide (KOH), sodium hydroxide (NaOH), or lithium hydroxide (LiOH), etc. Preferably the electrodes are contacted with sulphuric acid. Preferably, the electrode is contacted with between 0.1 and 5 M sulphuric acid, between 0.2 and 4 M sulphuric acid, between 0.3 and 3 M sulphuric acid, or between 0.4 and 2 M sulphuric acid. Most preferably, the electrode is contacted with 0.5 M sulphuric acid. Preferably, the sulphuric acid contacts the reference electrode, the working electrode and/or the counter electrode.

Preferably, detecting the electrochemical signal may comprise conductometric sensing, potentiometric sensing or amperometric sensing. Preferably, potentiometric sensing is utilized by measuring pulse wave voltammetry (PWV), square wave voltammetry (SWV) and/or cyclic voltammetry (CV).

Preferably, detecting the electrochemical signal comprises measuring the current across a range of potentials between −1V and 1V, or more preferably between −500 mV and 500 mV.

Furthermore, the means for detecting the electrochemical signal may be connected to a mobile or computer device, allowing a user to analyse the results of electrochemical detection. Accordingly, in one embodiment, the apparatus is connected to a mobile or computer device.

The target nucleic acid sequence may be a DNA or RNA sequence. The target nucleic acid sequence may be single-stranded or double-stranded, or a combination of single- and double-stranded. For example, the target nucleic acid sequence may be selected from chromosomal DNA, mitochondrial DNA, messenger RNA, transfer RNA, ribosomal RNA, small nuclear RNA, micro RNA, small-interfering RNA, viral RNA and extrachromosomal DNA.

In some embodiments, the target nucleic acid sequence may be from a virus, a bacteria, a mycoplasma, a fungus, an animal, a plant, an alga, a parasite, or a protozoan. Preferably, the target nucleic acid sequence is from a virus or a bacterium.

In some embodiments, the target nucleic acid sequence may be from an antibiotic resistant pathogen. Thus, it will be appreciated that the method according to the invention may be used to determine whether a subject is infected with an antibiotic resistant pathogen.

The virus may be a DNA virus or an RNA virus. The virus may be selected from one of the following: African horse sickness virus; African swine fever virus; Akabane virus; Bhanja virus; Caliciviruses (e.g., human enteric viruses such as norovirus and sapovirus), Cercopithecine herpesvirus 1; Chikungunya virus; Classical swine fever virus; coronaviruses (e.g., Severe Acute Respiratory Syndrome (SARS), Middle East Respiratory Syndrome (MERS), Severe Acute Respiratory Syndrome coronavirus 2 (SARS-CoV-2)); Dengue viruses such as serotypes 1 (DENV1) and 3 (DENV3), and related viruses such as the chikungunya virus (CHIKV); Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; flaviruses, Flexal virus; Foot and mouth disease virus; Germiston virus; Goat pox virus; Hantaan or other Hanta viruses; Hendra virus; human immunodeficiency virus (HIV); influenza viruses (e.g., H1N1, H5N1, Avian influenza virus); Lassa fever virus; Louping ill virus; Lymphocytic choriomeningitis virus; Poliovirus; Potato virus; pox viruses; South American hemorrhagic fever viruses; Variola major virus (Smallpox virus); Vesicular stomatitis virus; West Nile virus; Yellow fever virus; human-pathogenic flaviviruses such Zika virus.

The bacterium may selected from one of the following: Aeromonas hydrophila; Bacillus anthracis; Bacillus cereus; Botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium; Coxiella burnetii; Enterovirulent Escherichia coli group (EEC Group) such as Escherichia coli—enterotoxigenic (ETEC), Escherichia coli—enteropathogenic (EPEC), Escherichia coli —0157.'H7 enterohemorrhagic (EHEC), and Escherichia coli—enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Rickettsia prowazekii; Rickettsia rickettsii; Salmonella spp.; Schlerophthora rayssiae varzeae; Shigella spp.; Staphylococcus aureus; Streptococcus; Synchytrium endobioticum; Vibrio cholerae non-01; Vibrio cholerae 01; Vibrio parahaemolyticus and other Vibrios; Vibrio vulnificus; Xanthomonas oryzae; Xylella fastidiosa; Yersinia enterocolitica and Yersinia pseudotuberculosis; Yersinia pestis; Neisseria meningitidis; Neisseria gonorrhoea; or Klebsiella species, such as Klebsiella pneumoniae.

In another embodiment, the target nucleic acid sequence may be from a non-infectious disease, selected from the group consisting of: cancer; cardiovascular disease; chronic respiratory diseases; diabetes; autoimmune diseases; neurodegenerative diseases; and genetic diseases.

The sample is preferably a biological bodily sample taken from the test subject. Detecting the presence of the target nucleic acid sequence in the sample is therefore preferably carried out in vitro. The sample may comprise tissue, blood, plasma, serum, spinal fluid, urine, sweat, saliva, sputum, tears, breast aspirate, prostate fluid, seminal fluid, vaginal fluid, stool, cervical scraping, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The sample may be a biopsy.

In another embodiment, the sample may be contained within the test subject, which may be an experimental animal (e.g. a mouse or rat) or a human, wherein the method is an in vivo based test. Alternatively, the sample may be an ex vivo sample or an in vitro sample. Therefore, the cells being tested may be in a tissue sample (for ex vivo based tests) or the cells may be grown in culture (an in vitro sample). Preferably, the biological sample is an ex vivo sample.

In some embodiments, the method further comprises quantifying the target nucleic acid. The quantification step occurs after the amplification step of the method according to the first and second aspect. The quantification may comprise measuring the intensity of the electrochemical signal, to determine the concentration of the target nucleic acid in the sample.

The method may comprise the use of a positive control and/or a negative control against which the electrochemical signal may be compared. For example, a negative control sample is a sample which does not contain the target nucleic acid sequence. A positive control sample is a sample which does contain the target nucleic acid sequence.

Accordingly, in one embodiment, the method further comprises comparing the electrochemical signal with the electrochemical signal of a positive and/or negative control. In one embodiment, an increase in electrochemical signal compared to the negative control suggests that the sample does contain the target nucleic acid sequence. In another embodiment, an electrochemical signal with an intensity equal to or greater than the positive control suggests that sample does contain the target nucleic acid sequence.

Alternatively, a decrease in electrochemical signal compared to the positive control suggests that the sample does not contain the target nucleic acid. In another embodiment, an electrochemical signal with an intensity equal to or less than the negative control suggests that the sample does not contain the target nucleic acid sequence.

Advantageously, by detecting the presence of a target nucleic acid sequence, the methods of the invention provide a rapid and sensitive means for diagnosing both infectious and non-infectious diseases. Preferably, the methods of the invention are useful for enabling a clinician to make decisions with regards to the best course of treatment for a subject who is currently suffering from a disease. Preferably, the methods are useful for providing a prognosis of the subject's condition, such that treatment for the disease can be administered to the subject.

Accordingly, the difference in electrochemical signal between the sample and the positive and/or negative control, can be used as a diagnostic and/or prognostic marker, suggestive of the subject suffering from a disease. It will be appreciated that if the sample has an increased electrochemical signal compared to the negative control, or an electrochemical signal with an intensity equal to or greater than the positive control, then the subject suffers from the disease. Alternatively, if the sample has a decreased electrochemical signal compared to the positive control, or an electrochemical signal with an intensity equal to or less than the negative control, then the subject does not suffer from the disease.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, byway of example, to the accompanying Figures, in which:—

FIG. 1 illustrates the electrochemical detection principle by tracing phosphate ions generated during an amplification reaction (e.g. LAMP or PCR) to form molybdophosphate for signal output. Pyrophosphate (PPi) generated from the amplification reaction is hydrolysed by pyrophosphatase (PPase) into phosphate ions (Pi), which further react with sodium molybdate to form molybdophosphate on an electrode surface. The electrochemical response of the generated molybdophosphate can then be detected for target quantitative analysis with high sensitivity.

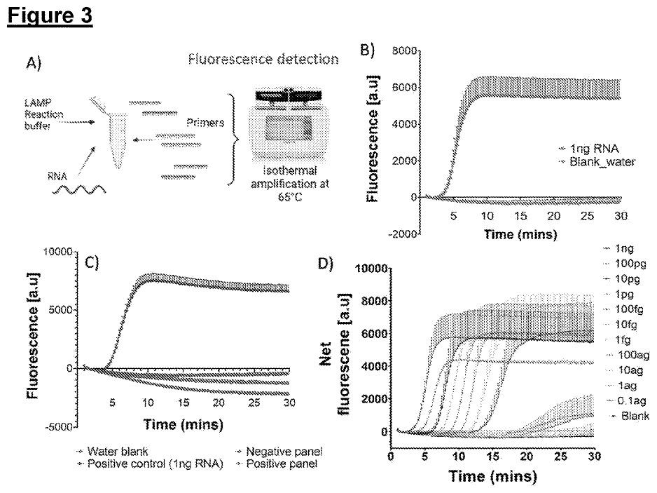

FIG. 2 illustrates the principle of electrochemical nucleic acid detection with the M gene (membrane protein) of SARS-CoV-2.

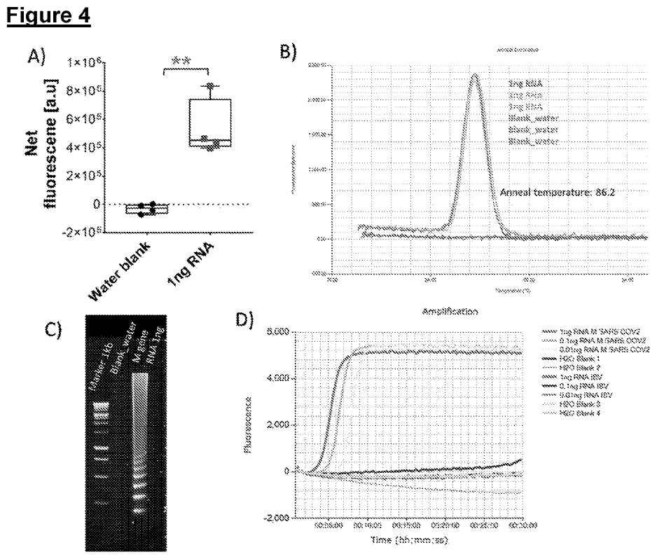

FIG. 3 shows the development of SARS-CoV-2 fluorescent LAMP. A) Schematic of fluorescent LAMP detection. Six LAMP primers comprising of outer F3/B3, looping inner FIP/BIP and loop primers LF/LB, Bst polymerase, RT reverse transcriptase and isothermal reaction buffer was used for the LAMP reaction. The amplification was carried out isothermally at 65° C. for 30 minutes. Real time fluorescence was monitored over 30 minutes. B) SARS-CoV-2 M gene RNA detection with fluorescent LAMP. Values are mean f Standard error of the mean (SEM) for both positive (1 ng RNA templates) and blank water (n=4). C) Cross specificity test for M gene LAMP. Positive panel includes DNA and RNA from 21 respiratory pathogens. Negative panel includes inactivated DNA and RNA from the same 21 respiratory pathogens. Values are mean f SEM (n=4). D) LOD analysis for M gene fluorescent LAMP. Values are mean±SEM (n=4).

FIG. 4 shows A) M gene fluorescent LAMP using water blank and 1 ng RNA as a template. B) Anneal derivative of M gene LAMP at 86° C. X-axis showing temperature (° C.) and y-axis fluorescence (arbitrary units) for the anneal derivative. C) 1% agarose gel showing positive (1 ng RNA) and negative (blank water) LAMP reactions. D) Cross specificity test of M gene fluorescent LAMP using avian IBV coronavirus. Values are mean±SEM (n=3).

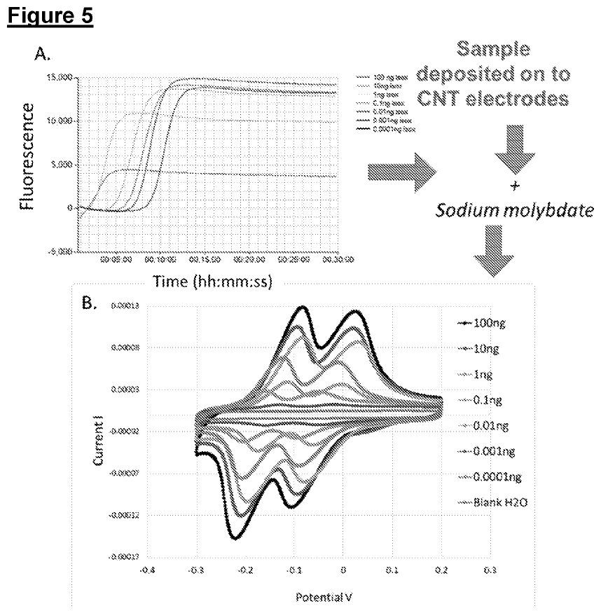

FIG. 5 illustrates the detection of M gene LAMP products using SARS-CoV-2 RNA as template (A. fluorescence detection). The sample was deposited onto a carbon nanotube (CNT) electrode with sodium molybdate, and the positive and negative current (I) was measured across a range of potentials (V) (B).

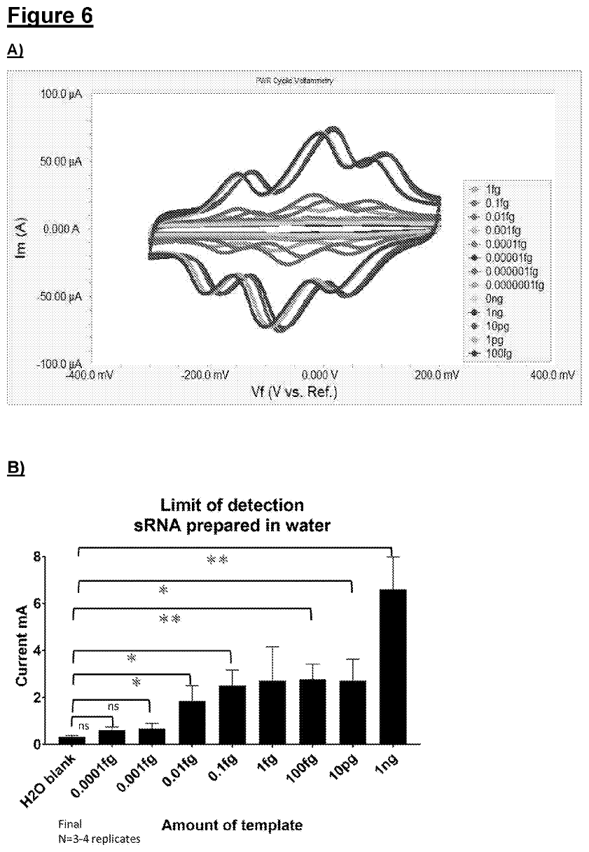

FIG. 6 shows the LOD analysis for M gene using the electrochemical LAMP assay. A. Current vs. potential (V) plot of signals from templates ranging from 1 ng to 0.0000001fg. B. Average current calculations for various samples with templates ranging from 1 ng to 0.0001fg

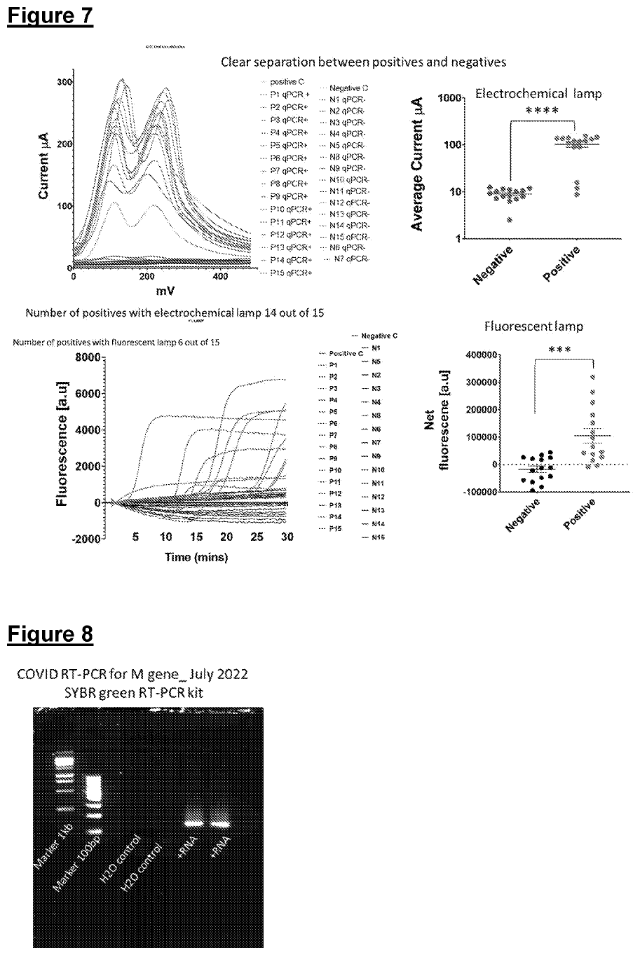

FIG. 7 illustrates a comparison between electrochemical LAMP and fluorescent LAMP detection of SARS-CoV-2 M gene in clinical samples.

FIG. 8 shows detection for SARS-CoV-2 by regular RT-PCR, using 2.5% agarose gel electrophoresis. Positive amplifications show a band of ~135 bp.

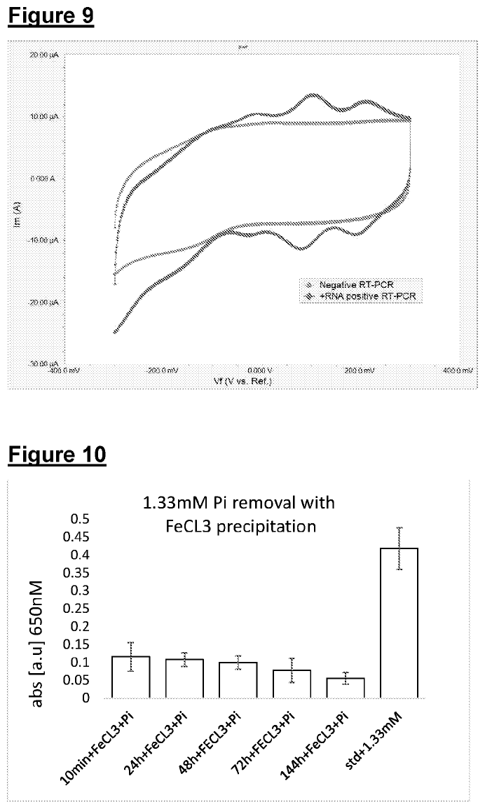

FIG. 9 shows electrochemical detection of SARS-CoV-2 on CNT screen printed electrodes. The positive electrochemical signal is shown as red and negative as grey.

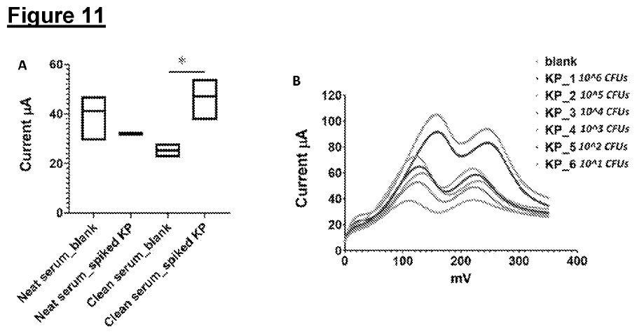

FIG. 10 illustrates that phosphate removal can be achieved by FeCl₃ precipitation. Molybdenum blue phosphate reaction was used to detect phosphates in a standard KH2PO4 solution after phosphate removal. Phosphate was removed (precipitated) for 10 minutes at the start of the reaction. The precipitation reaction was carried on for 144 hours.

FIG. 11 shows (A) Electrochemical detection of K. pneumoniae LAMP in neat and phosphate removed serum. Values are mean G SEM (n=3-4). (B) Sensitivity analysis of K. pneumoniae LAMP. Live K. pneumoniae with different dilutions were spiked into phosphate removed human serum. The test was sensitive to detecting as low as 10 colony forming units. Statistical was done using one-way ANOVA and Tukey's and unpaired t-test; *, p<0.05; **, p<0.005.

### EXAMPLES

### Materials and Methods

### Selection of Suitable Gene Targets for Detection of SARS-CoV-2 by LAMP

### Identification of Target Gene for SARS-CoV-2

The inventors identified membrane protein (M) gene of SARS-CoV-2 using comparative genomics as described in Poirier et al. 2021 [1].

M membrane glycoprotein [Severe acute respiratory syndrome coronavirus 2]:

[SEQ ID No: 1]
ATGGCAGATTCCAACGGTACTATTACCGTTGAAGAGCTTAAAAAGCTCCTTGAACAATGGAACCTAGTAA
TAGGTTTCCTATTCCTTACATGGATTTGTCTTCTACAATTTGCCTATGCCAACAGGAATAGGTTTTTGTA
TATAATTAAGTTAATTTTCCTCTGGCTGTTATGGCCAGTAACTTTAGCTTGTTTTGTGCTTGCTGCTGTT
TACAGAATAAATTGGATCACCGGTGGAATTGCTATCGCAATGGCTTGTCTTGTAGGCTTGATGTGGCTCA
GCTACTTCATTGCTTCTTTCAGACTGTTTGCGCGTACGCGTTCCATGTGGTCATTCAATCCAGAAACTAA
CATTCTTCTCAACGTGCCACTCCATGGCACTATTCTGACCAGACCGCTTCTAGAAAGTGAACTCGTAATC
GGAGCTGTGATCCTTCGTGGACATCTTCGTATTGCTGGACACCATCTAGGACGCTGTGACATCAAGGACC
TGCCTAAAGAAATCACTGTTGCTACATCACGAACGCTTTCTTATTACAAATTGGGAGCTTCGCAGCGTGT
AGCAGGTGACTCAGGTTTTGCTGCATACAGTCGCTACAGGATTGGCAACTATAAATTAAACACAGACCAT
TCCAGTAGCAGTGACAATATTGCTTTGCTTGTACAGTAA

Briefly, assembly metrics were obtained using Quast version 4.5 [2] and genome assemblies were annotated using Prokka V.1.14.5 and pangenome using Roary version with a 95% identity cut-off. [3]. The inventors targeted membrane (M) glycoprotein which is an abundant structural protein in Coronaviruses and plays an important role in RNA packaging [4, 5].

1654 SARS-CoV-2 genomes were downloaded from GISAID (27 Mar. 2020). These genomes represented all sequenced SARS-CoV-2 genomes at the time of access. Nucleotide variation of each ORF was determined using BLAST v2.12. Alignment of representative ORFs performed using MUSCLE v3.8 showed that the N-gene, ORF7a, and M-gene encoding structural proteins had a high degree of nucleotide conservation. LAMP primers to specifically target conserved regions of each of these ORFs were designed using LAMP Designer (Optigene, UK). Alignment of the N-gene, ORF7a, and M-gene to the corresponding structural genes in representative SARS, MERS and bat SARS-like coronavirus genomes to evaluate potential cross-reactivity was performed using MUSCLE v3.8

### SARS-CoV-2 Membrane (M) Protein Synthetic RNA Synthesis

A synthetic plasmid encoding the SARS-CoV-2 M gene (Wuhan-Hu-1 isolate, GenBank accession number NC_045512.2) was purchased from Integrated DNA Technologies. The M gene was first PCR amplified to introduce a T7 promoter in the 5′UTR using the following primers 5′-TAATACGACTCACTATAGTAATCAGACAAGGAACTGATTA-3′ (SEQ ID No: 2) and 5′-CGAAGGTGTGACTTCCATG-3′ (SEQ ID No: 3). M gene transcript RNAs were then directly transcribed in vitro using the T7 RNA polymerase run-off reactions from PCR products containing the T7 polymerase promoter sequence and purified as previously described [6].

### RNA Extraction

RNA extraction was performed on 100 μL of VTM using the commercial QIAgen RNeasy kit (Qiagen, Valencia, CA, USA), according to manufacturer instructions.

### qRT PCR Materials and Methods for the Clinical Samples

The inventors used the CDC 2019-Novel Coronavirus (2019-nCoV) Real-Time RT-PCR Diagnostic Panel (Centers for Disease Control and Prevention, Division of Viral, Atlanta USA), containing the 2019-nCoV_N1, 2019-nCoV_N2 and Human RNase P combined primers and probes mix, as per the Instructions for Use (fda.gov). The 25-μl RT-qPCR reactions consisted of 12.5 μl 2× Reaction Mix, 0.2 μM of each primer, and 0.1 μM probe, 0.5 μl of SuperScript® III RT/Platinum® Taq Mix, and 2 μL of RNA (extracted from clinical samples or synthetic). The amplification process was performed in the CFX96 Touch Real-Time PCR Detection System (BioRad Laboratories, Watford, UK), according to the cycling protocol. The amount of viral RNA in each sample was estimated by comparing the cycle threshold values (Ct) to the standard curve made by serial 10-fold serial dilutions of synthetic RNA.

### Specificity Testing Panel for M LAMP Primers

The designed LAMP primer set was validated for its analytical specificity by testing cross-reactivity against other respiratory pathogens: Adenovirus Type 6, Bordetella pertussis and parapertussis, Chlamydia pneumoniae, Coronaviruses 229E HKU1, NL63, OC43 surrogates, Human Metapneumovirus surrogate, Human Rhinovirus, Influenza A (subtypes H1, Hi-2009 and H3) and Influenza B, Mycoplasma pneumoniae, Parainfluenza Viruses (1, 2, 3, and recombinant 4a) and Respiratory Syncytial Virus (using the Respiratory (21 Targets) Control Panel, Microbiologics Minnesota, USA).

TABLE 1
Primers

| | | | SEQ |
|---|---|---|---|
| Name | Quality | Primer | ID No |
| F3 | | TTGCTATCGCAATGGCTT | 4 |
| B3 | | TGATGTCACAGCGTCCTA | 5 |
| FIP(F1c + F2) | | GGAGTGGCACGTTGAGAAGAATGCTTCTTTCAGACTGTTTGC | 6 |
| BIP(B1c + B2) | | GGCACTATTCTGACCAGACCGGATGTCCACGAAGGATCAC | 7 |
| LoopF | | GGATTGAATGACCACATGGAAC | 8 |
| LoopB | | AGTGAACTCGTAATCGGAGC | 9 |
| F2 | | GCTTCTTTCAGACTGTTTGC | 10 |
| F1c | | GGAGTGGCACGTTGAGAAGAAT | 11 |
| B2 | | GATGTCCACGAAGGATCAC | 12 |
| B1c | | GGCACTATTCTGACCAGACCG | 13 |
| Product | Best | Sequence Length: 669 | |

### Reverse Transcriptase (RT)-Loop-Mediated Isothermal Amplification (LAMP)

LAMP reaction buffer was designed to achieve maximum amplification along with minimum interference in electrochemical detection. The buffer composition for a 25 μL reaction includes 1× isothermal amplification (buffer containing 20 mM Tris-HCl, 10 mM ammonium sulphate (NH₄)₂SO₄, 50 mM potassium chloride KCl, 2 mM magnesium sulphate MgSO₄, 0.1% Tween® 20, (pH 8.8 @25° C.)) (NEB), 6 mM MgSO₄ (NEB), 1.4 mM dioxyribonucleotides dNTPs (NEB), 1.6 μM FIP+BIP primers (Merck), 0.2 μM F3/B3 primers (Merck), 0.4 M loop F/B primers (Merck), 320U/mL Bst 2.0 DNA polymerase (NEB), 7500 U/mL warmStart® RTx reverse transcriptase (NEB), 2000 U/mL thermostable inorganic pyrophosphatase (NEB).

| | |
|---|---|
| Buffer | Added to 25 uL reaction |

| 10X isothermal amplification buffer | 1X | |
|---|---|---|
| MgSO4 | 5 | mM |
| DMSO | 2.50% | |
| dNTPs | 2.5 | mM |
| Bst polymerase | 1 | U |
| Phosphatase | 1 | U |
| transcriptase | 7.5 | U |
| water | as required | |
| template sRNA | 10 | ng/uL |

SYBR green dye was used for fluorometric LAMP detection and IBV Beau-R RNA [7], was extracted from virus stocks using RNeasy columns (QIAgen), following the manufacturer's instructions, and including on-column DNAse treatment using an RNase-free DNase set (QIAgen).

### Electrochemical Detection

Phosphomolybdate detection for electrochemical detection was done using a single step reaction. Briefly, 25 μL LAMP products were mixed with 25 μL of 20 mM sodium molybdate and mixed by pipetting and were incubated for 10 minutes. Screen-printed carbon electrodes modified with carboxylic functionalised carbon nanotube (CNT) (110-D) were purchased from Metrohm. CNT electrodes were rinsed with ultra-pure water and primed with 0.1M sodium nitrate for 3-10 cycles. 5-10 μL of LAMP and sodium molybdate mix was placed on the electrode and dried in an oven at 50° C. for 3-5 minutes. Dried electrodes were rinsed with ultra-pure water and connected to the GAMRY potentiostat electrode holder. Around 200 μL of 0.5M sulphuric acid was dropped onto electrode to cover the reference, counter and working electrodes. Pulse wave, square wave and cyclic voltammetry were setup as a sequence with measurement parameters. Data analysis was done using GAMRY EChem analyst software, Excel and Origin.

### Electrochemical Detection of Synthetic or Simulated Clinical Samples

Biological fluids or clinical samples will inherently have phosphate ions which will interfere with the electrochemical detection. To avoid phosphate interference and reduce noise, the inventors performed a sample clean up step with profoldin phosphate removal columns. Briefly, live bacteria or RNA were spiked into serum (procured from a healthy donor); serum was cleaned with phosphate removal columns prior to DNA/RNA extraction.

### Phosphate Removal

Phosphate Removal Columns were Purchased from ProFoldin (https://www.profoldin.com/phosphate_removal.html). To remove phosphates, serum and saliva samples were treated with phosphate removal columns as described by the manufacturer (https://www.profoldin.com/f/P154-_Micro_Phosphate_Remroval_Column_Set.pdf. The pre-packed phosphate removal column was spun using a bench-top microcentrifuge to set down the resin. The caps of 1.5 ml-eppendorf tubes were cut off and the tubes were used as receivers of the columns. The column bottom tips and caps were removed and placed into 1.5 ml-eppendorf tubes. Columns were spun at 13,000 rpm for 1 min and solution was discarded. The columns were spun at 13,000 rpm for 1 min again to make sure the resin is almost dry. Each column were transferred into a clean labelled 1.5-ml eppendorf tube. 200 μL of the sample was loaded onto each column and spun at 1000 rpm for 1 min. Then columns were spun again at 13,000 rpm for 1 min and the elute was saved. The elute was spun at 13,000 rpm for 1 min to remove any insoluble material to remove any insoluble material.

Ferric trichloride hexahydrate (FeCl3) was used for phosphate precipitation assays (8). 100 mL type 1 water was used as the base. 1 uM and 1.33 mM potassium phosphate (KH₂PO₄) were added to the water from a stock solution of 1M KH₂PO₄. Two precipitation assays were setup at room temperature, one with 1 uM KH₂PO₄ as the starting amount and the second with 1.33 mM KH₂PO₄ as the starting phosphate amount. 2.47 mM FeCl3 was added to the KH₂PO₄ solutions. The solutions were stirred. The pH was adjusted to 7.0 for both precipitation assays. The solutions were withdrawn at various times from the start of the assays, for example, 20 minutes, 24 h, 48 h, 72 h and 144 h. Withdrawn samples (~2 mLs) were centrifuged at 13000 rpm for 5 minutes (after spinning a red pellet appears) and supernatant were filtered using a 0.45 u syringe filter. The amount of phosphate that remained in the filtered supernatant was measured using colorimetric molybdenum blue assay.

The molybdenum blue reaction was used for the determination of orthophosphate in LAMP reactions and neat serum and saliva samples treated with and without the phosphate clean up step. The principle of this reaction involves the formation of a polyoxometallate species, a heteropoly acid, from orthophosphate and molybdate under acidic conditions, which when reduced forms an intensely coloured phosphomolybdenum blue species. Working solutions of sulphuric acid (11N/5.4M), ascorbic acid (6% w/v), ammonium molybdate tetrahydrate (1.26% w/v)-potassium antimony tartrate trihydrate (0.21% w/v) and potassium phosphate monobasic (0.1M) were prepared in distilled water from concentrated stock solutions. Potassium dihydrogen phosphate (KH₂PO₄) standards were prepared in a range of dilutions in distilled water. Samples were diluted to 5 mL with distilled water. 0.1 mL of working solution of sulphuric acid, 0.4 mL ammonium molybdate-potassium tartrate and 0.2 mL ascorbic acid solution working solution were added to the samples. The reaction was incubated at room temperature for 5 minutes. 1 mL of the reaction was transferred to a plastic cuvette and absorbance was measured at 650 nm using a spectrophotometer.

### RT-PCR

SYBR green RT-PCR kit was used to amplify ~135 bp of M gene using synthetic RNA as the template. The PCR mix was prepared according to manufacturer's instructions. A final concentration of 1× SYBR Green Taq Ready mix, 3 mM MgCl2, 10 ng RNA template, 0.5 uM forward and reverse primers, 1 unit/uL MLV-RT. The reaction includes: Reverse transcription at 44° C. for 30 minutes, Initial denaturation 94° C. for 2 mins, 40 cycles of denaturation at 94° C. for 15 sec, 60° C. for 1 min, Extension 72° C. for 0.5 min, final extension 72° C. for 2 mins.

### Primer Sequences for RT-PCR:

Forward primer sequence:
(SEQ ID No: 14)
TCTTCTCAACGTGCCACTCC
Reverse primer sequence:
(SEQ ID NO: 15)
CCTTGATGTCACAGCGTCCT

### Results and Discussion

### Phosphate Removal

The inventors were surprised to discover that when performing the electrochemical assay on clinical samples, the presence of free phosphate ions was leading to false positive results. Accordingly, the inventors set out to test whether phosphate removal from the samples can be achieved by FeCl₃ precipitation. Molybdenum blue phosphate reaction was used to detect phosphates in a standard KH₂PO₄ solution after phosphate removal. Phosphate was removed (precipitated) for 10 minutes at the start of the reaction, and the precipitation reaction was carried on for 144 hours. As illustrated in FIG. 10, the inventors successfully demonstrated that phosphate removal can be achieved by simple FeCl₃ precipitation.

### Development of SARS-CoV-2Fluorescent LAMP

The inventors targeted membrane protein (M) gene of SARS-CoV-2 to design a fluorescent LAMP based assay. Six primers: forward and backward loop primers (LF, LB), forward and backward outer and internal primers (F3, B3, FIP, BIP) were designed to amplify 669 bp of M gene (See Table 1 for primers). A defined LAMP reaction buffer (see materials and methods) was developed to obtain optimal DNA amplification and to minimise any cross reactivity from the buffer components (such as potassium chloride KCL and dioxyribonucleotides dNTPs) with the electrochemical detection used in the reaction buffer. The inventors used 50 mM KCl and 1.4 mM dNTPs which caused no detectable interference with the electrochemical LAMP assay. The inventors used synthetic M gene RNA to develop the LAMP test (see Materials and Methods).

The fluorescence detection for M gene targeted LAMP assay is shown in FIG. 3B. The inventors validated the specificity of M gene targeted LAMP assay by checking the cross specificity of the primers using multiple DNA and RNA as template from multiple respiratory pathogens and avian IBV coronavirus (FIG. 3C). The cross-specificity test demonstrated 100% specificity of the primers and the LAMP assay for SARS-CoV2 M gene. The inventors calculated the limit of detection (LOD) for the fluorescence-based assay. A range of dilutions of up to 10⁻¹⁰ (0.1 attograms) were prepared using 1 ng synthetic RNA in water as the starting template. The LOD of fluorescent assay was recorded to be 10⁻⁷ (100 attograms) which accounts for ~264 copies of DNA per reaction (FIG. 3D). The inventors used this working fluorescent LAMP assay as a reference test for the development of SARS-CoV2 electrochemical LAMP.

### Development of SARS-CoV-2 Electrochemical LAMP

A typical LAMP reaction generates pyrophosphates which is proportional to the amount of amplified DNA. The inventors used thermostable pyrophosphatase in the reaction buffer, which cleaves pyrophosphates into phosphates (Pi) during the LAMP reaction. The phosphates when added to sodium molybdate produce phosphomolybdate, which in turn undergoes oxidation and reduction reactions and generates electric current under acidic conditions. The schematic of the electrochemical LAMP workflow is shown in FIG. 2.

The electrochemical detection for M gene targeted LAMP assay is shown in FIG. 5. The LAMP products were deposited onto a CNT modified carbon electrode with sodium molybdate, and the positive and negative current (I) was measured across a range of potentials (V).

The inventors calculated the limit of detection (LOD) for the electrochemical-based assay. A range of dilutions of up to 10⁻¹⁰ (0.1 attograms) were prepared using 1 ng synthetic RNA in water as the starting template. The LOD of the electrochemical assay was recorded to be 0.01 fg, which accounts for ~26 copies of DNA per reaction (FIG. 6).

Next, the inventors compared the detection of SARS-CoV-2 M gene in clinical samples, using both the electrochemical LAMP assay and the fluorescent LAMP assay. As shown in FIG. 7, there is a clear separation between positive and negative samples when using the electrochemical based assay, which was able to detect 14 out of the 15 positive samples. In contrast, the fluorescent based assay was only about to detect 6 out of 15 of the positive samples.

### Development of SARS-CoV-2 Electrochemical RT-PCR

A typical RT-PCR reaction generates pyrophosphates which is proportional to the amount of amplified DNA. The inventors used thermostable pyrophosphatase in the reaction buffer, which cleaves pyrophosphates into phosphates (Pi) during the PCR reaction. The phosphates when added to sodium molybdate produce phosphomolybdate, which in turn undergoes oxidation and reduction reactions and generates electric current under acidic conditions.

As illustrated in FIG. 8, regular RT-PCR was used to detect SARS-CoV-2 by 2.5% agarose gel electrophoresis, and positive amplifications show a band of ~135 bp. The RT-PCR products were also deposited onto a CNT based electrode with sodium molybdate, and the electrochemical signal was measured. As illustrated in FIG. 9, the inventors demonstrated that electrochemical detection also works successfully for detecting RT-PCR products.

Development of K. pneumoniae Electrochemical LAMP

Additionally, the inventors checked the versatility of the electrochemical-rapid diagnostic test (RDT) for the detection of Klebsiella pneumoniae spiked into human serum. This serum was treated for phosphate removal as discussed for the SARS-CoV-2 test. The inventors targeted the K. pneumoniae yhaI gene as described in Poirier et al., 2022.

Live bacteria were spiked into human serum followed by cell lysis and DNA extraction. FIG. 11A shows the electrochemical detection of K. pneumoniae spiked in neat vs. phosphate removed (clean) serum. As illustrated, there was a significant increase in sensitivity of K. pneumoniae detection in clean serum. The KP electrochemical assay was sensitive to detecting as low as 10 viable colony forming units per mL (FIG. 11B). Accordingly, these data demonstrate the suitability of the electrochemical assay for both bacterial and viral diagnostics.

### CONCLUSIONS

The inventors have established an accurate and sensitive nucleic acid detection method, by optimising an electrochemical assay for use with clinical samples. In particular, the inventors surprisingly discovered that the removal of phosphate ions from clinical samples prior to performing nucleic acid amplification and detection, increased the accuracy of the assay and significantly reduced the occurrence of false positives. Additionally, the inventors have demonstrated that this electrochemical detection works when using different nucleic acid amplification methods, including both LAMP and PCR. Accordingly, the inventors have established an improved, accurate and sensitive method for detecting the presence of pathogens or genetic diseases in clinical samples, or determining whether pathogens are drug resistant. This novel electrochemical approach therefore provides a new avenue for accurate nucleic acid detection.

### REFERENCES

1. Poirier et al. 2021
2. doi: 10.1093/bioinformatics/btto86
3. doi: 10.1093/bioinformatics/btv421
4. doi:10.116/j.bbadis.2020.165878
5. doi:10.1016/j.jmii.2020.03.022
6. doi: 10.1093/nar/gkg1118
7. doi: 10.1128/JVI.75.24.12359-12369.2001
8. doi:10.116/j.jhazmat.2009.12.018
9. doi.org/10.116/j.psep.2015.03.011
10. doi.org/10.1111/j.1574-6976.1994.tb00131.x

1. A method of detecting a target nucleic acid sequence in a sample obtained from a subject, the method comprising:
removing substantially all free phosphate ions from a sample comprising a target nucleic acid sequence;
amplifying the target nucleic acid sequence in the sample to generate amplified products;
using the amplified products to create an electroactive compound; and
detecting an electrochemical signal of the electroactive compound, wherein an electrochemical signal indicates the presence of the target nucleic acid sequence in the sample.

2. A method of diagnosing or prognosing a disease in a subject, the method comprising detecting a target nucleic acid sequence in a sample obtained from a subject, by:
removing substantially all free phosphate ions from a sample comprising a target nucleic acid sequence;
amplifying the target nucleic acid sequence in the sample to generate amplified products;
using the amplified products to create an electroactive compound; and
detecting an electrochemical signal of the electroactive compound, wherein an electrochemical signal indicates that the subject suffers from a disease, is infected by a targeted pathogen, or has a negative prognosis.

3. An apparatus for detecting a target nucleic acid sequence in a sample obtained from a subject, the apparatus comprising:
means for removing substantially all free phosphate ions from a sample comprising a target nucleic acid sequence;
means for amplifying the target nucleic acid sequence in the sample to generate amplified products;
means for creating an electroactive compound using the amplified products; and
means for detecting an electrochemical signal of the electroactive compound.

4. The method according to claims 1 and 2, or the apparatus according to claim 3, wherein removing substantially all free phosphate ions from the sample comprises removing at least 50%, at least 60%, or at least 70% of the free phosphate ions from the sample, or more preferably removing at least 80%, at least 90%, or at least 95% of the free phosphate ions from the sample.

5. The method or apparatus according to any preceding claim, wherein removing substantially all free phosphate ions from the sample comprises the use of:
i) phosphate-removing bacteria;
ii) selective absorption;
iii) ferric trichloride hexahydrate (FeCl3) precipitation; and/or
iv) a phosphate removal column.

6. The method according to any preceding claim, wherein the method further comprises measuring the concentration of free phosphate ions in the sample after substantially all the free phosphate ions have been removed.

7. The method according to claim 6, wherein the method comprises detecting free phosphate ions using the molybdenum blue phosphate reaction.

8. The method according to any preceding claim, wherein amplifying the target nucleic acid sequence in the sample comprises amplification by polymerase chain reaction (PCR), loop-mediated isothermal amplification (LAMP), recombinase polymerase amplification (RPA), or nucleic acid sequence-based amplification (NASBA).

9. The method according to claim 8, wherein amplifying the target nucleic acid sequence in the sample comprises amplification by polymerase chain reaction (PCR) or loop-mediated isothermal amplification (LAMP).

10. The method according to any preceding claim, further comprising contacting the amplified products with pyrophosphatase (PPase) to generate phosphate ions (Pi).

11. The method according to any preceding claim, wherein using the amplified products to create an electroactive compound comprises contacting the amplified products with a phosphate-precipitating salt to produce phosphomolybdate.

12. The method according to claim 11, wherein the phosphate-precipitating salt comprises or is a molybdate, more preferably a Group I metal molybdate.

13. The method according to either claim 11 or claim 12, wherein the phosphate-precipitating salt is sodium molybdate.

14. The method according to any preceding claim, wherein the electrochemical signal is conducted along an electrode.

15. The method according to any preceding claim, wherein the electrochemical signal is detected using a two electrode system comprising a working electrode (WE) and a counter electrode (CE), or wherein the electrochemical signal is detected using a three electrode system comprising a WE, a CE and a reference electrode (RE).

16. The method according to either claim 14 or claim 15, wherein the electrode is a carbon based screen-printed electrode, more preferably a carbon nanotube (CNT) modified carbon based screen-printed electrode, and even more preferably an acid or O₂ plasma functionalised CNT modified carbon-based screen printed electrode.

17. The method according to any one of claims 14 to 16, wherein the electrode is connected to a potentiostat for measuring the electrochemical signal.

18. The method according to any one of claims 14 to 17, further comprising contacting the electrode with an electrolyte.

19. The method according to claim 18, wherein the electrolyte is selected from the group consisting of nitrate, borate, halides, phosphate, phosphonates, carbonate, sulphate, sulphuric acid (H₂SO₄), phosphoric acid (H₃PO₄), perchloric acid (HClO₄), potassium hydroxide (KOH), sodium hydroxide (NaOH), and lithium hydroxide (LiOH)

20. The method according to any preceding claim, wherein detecting the electrochemical signal comprises conductometric sensing, potentiometric sensing or amperometric sensing.

21. The method according to claim 20, wherein potentiometric sensing is utilized by measuring pulse wave voltammetry (PWV), square wave voltammetry (SWV) and/or cyclic voltammetry (CV).

22. The method according to any preceding claim, wherein detecting the electrochemical signal comprises measuring the current across a range of potentials between −1V and 1 V, or more preferably between −500 mV and 500 mV.

23. The apparatus according to any preceding claim, wherein the apparatus is connected to a mobile or computer device.

24. The method or apparatus according to any preceding claim, wherein the target nucleic acid sequence is a DNA or RNA sequence, optionally wherein the target nucleic acid sequence is selected from chromosomal DNA, mitochondrial DNA, messenger RNA, transfer RNA, ribosomal RNA, small nuclear RNA, micro RNA, small-interfering RNA, viral RNA and extrachromosomal DNA.

25. The method or apparatus according to any preceding claim, wherein the target nucleic acid sequence is from a virus, a bacteria, a mycoplasma, a fungus, an animal, a plant, an alga, a parasite, or a protozoan, or wherein the target nucleic acid sequence is from an antibiotic resistant pathogen.

26. The method or apparatus according to any one of claims 1 to 24, wherein the target nucleic acid sequence is from a non-infectious disease selected from the group consisting of: cancer; cardiovascular disease; chronic respiratory diseases; diabetes; autoimmune diseases; neurodegenerative diseases; and genetic diseases.

27. The method according to any preceding claim, further comprising measuring the intensity of the electrochemical signal, to determine the concentration of the target nucleic acid in the sample.

28. The method according to any preceding claim, further comprising comparing the electrochemical signal with the electrochemical signal of a positive and/or negative control.
